(19) United States
(12) Patent Application Publication
(10) Pub. No.: US 2012/0282623 A1
(43) Pub. Date: Nov. 8, 2012

(54) RAPID PATHOGEN DETECTION TECHNIQUES AND APPARATUS

(75) Inventors: Taku Murakami, Irvine, CA (US); Toshit Sen, Chandicarch (IN)

(73) Assignees: Hitachi Chemical Research Center, Inc., Irvine, CA (US); Hitachi Chemical Co., Ltd., Shinjuku-ku, Tokyo (JP)

(21) Appl. No.: 13/574,296

(22) PCT Filed: Jan. 24, 2011
(86) PCT No.: PCT/US11/00123

§ 371 Date: Jul. 23, 2012

Related U.S. Application Data
(60) Provisional application No. 61/297,616, filed on Jan. 22, 2010.
(60) Provisional application No. 61/301,537, filed on Feb. 4, 2010.

(51) Int. Cl. C12Q 1/04; C12Q 1/68; G01N 33/569

(52) U.S. Cl. 435/6.12; 435/34; 435/7.1

(57) ABSTRACT

Methods for selectively detecting a live pathogen in a sample containing live and dead pathogens, without detecting the dead pathogen are disclosed. Such methods may include: (i) immobilizing at least a portion of the live and dead pathogens on a solid support with a physical barrier; (ii) incubating the solid support in a growth medium, where the live pathogen can multiply and the multiplied pathogen move from the solid support to a supernatant of the growth medium; and (iii) detecting the multiplied pathogen in the supernatant by a pathogen assay.

### FIELD

The present disclosure relates to methods, and apparatus for rapidly detecting pathogens in large volume particulate samples.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

According to a recent estimate by the Centers for Disease Control (CDC), food-borne pathogens are the cause of 76 million cases of food-borne illness, 325,000 hospitalizations, and 5,000 deaths within the United States alone each year. In addition to the harmful medical effects on humans, these food-borne outbreaks have detrimental economic effects due to medical costs, lost productivity, product recalls, and halted exports. The USDA recommends a zero-tolerance policy for certain food-borne pathogens, as even a small amount of pathogen can survive and cause food poisoning or even an outbreak. Therefore, the development of sensitive pathogen detection technology capable of detecting even a single pathogen in food samples is necessary.

Many detection technologies and products are currently available for the detection of food-borne pathogens, but it is still challenging to detect a single pathogen contaminating a few grams of food sample within a 24 hour time frame. Generally, pathogens are extracted from a food sample by dilution and homogenization, resulting in a sample volume of as much as a few hundred mL, or even larger. In order to detect small numbers of pathogens in a large volume of sample, it is necessary to culture the organisms until the pathogen concentration reaches a level suitable for a pathogen detection assay. The necessary pre-enrichment time depends on the doubling time, the viability of the target pathogen, the pathogen concentration required for detection, and the potential growth inhibitory effect of food samples. Pre-enrichment usually takes at least 12 to 48 hours. Therefore, even using highly sensitive detection assays such as polymerase chain reaction (PCR) and enzyme-linked immunosorbent assay (ELISA), the detection of pathogen contamination in a food sample still requires one to two days. Additionally, these assays are generally expensive and require skilled laboratory personnel.

Furthermore, food pathogen assays with cultural pre-enrichment require handling enriched pathogen, which may cause secondary contamination of laboratories and personnel. Thus, these assays are not appropriate for on-site testing at food manufacturing plants and may instead need to be conducted off-site at, for example, reference laboratories. Also, cultural pre-enrichment will cause the loss of quantitative information regarding contaminated food pathogens, because the viability and contamination level of pathogens and the ingredients of food samples can affect pathogen growth rate and time to reach the stationary phase or the decline phase of organism growth. Therefore, food pathogen assays with pre-enrichment are non-quantitative and estimating the level of food pathogen contamination is difficult.

In order to overcome the aforementioned disadvantages of pathogen assays with pre-enrichment, alternative enrichment procedures such as centrifugation or filtration of a large sample volume to increase the pathogen concentration may be necessary. However, centrifugation requires a centrifuge and is generally a cumbersome process, especially when a large number of large volume samples need to be tested.

Filtration may be performed in higher throughput and can concentrate even a low number of pathogens present in large volume samples. To concentrate pathogens, a filtration method generally utilizes microfiltration (MF) membrane filters, which generally have pore sizes smaller than the target pathogen. Thus, pathogens can be concentrated on such filters and detected by several detection methods, such as colony formation and DNA probe hybridization. However, these membrane filters have very low particulate-holding capacities and tend to readily get clogged with particulate food extracts due to their small pore sizes. These filters are acceptable for samples containing less particulate, such as drinking or environmental water, or for very small volumes of particulate samples. Depth filters with an appropriate retention rate are also used to trap pathogens; however, the detection of pathogens trapped in such depth filters is complicated by their three-dimensional matrix structure and filter thickness.

It is very important to detect live pathogens in food samples. Polymerase chain reaction (PCR) is becoming a new gold standard for rapid pathogen detection because of its assay sensitivity, speed and accuracy. However, it is very difficult to distinguish live and dead pathogens by PCR itself because PCR targets short specific regions of the pathogen genomic DNA, which often remain intact even after organism death. Currently, in the food industry, PCR assay is often combined with overnight pre-enrichment step in a growth medium, where live pathogens can grow while dead pathogens do not.

Because PCR is very sensitive and able to detect even less than 10 copies of genomic DNA, in the case when food samples are highly contaminated with dead pathogens only, PCR assay will result in false positive results, causing delay of product manufacturing and shipment without necessity. Such a situation could occur, for example, in the case of assaying pasteurized food samples such as deli meat, milk and orange juice if they are contaminated with pathogen before pasteurization. In order to avoid this issue, it is necessary to use a pre-enrichment condition where live pathogens can grow to much higher concentrations than any possible contamination levels of dead pathogens in food samples. For example, 1 cfu live Listeria monocytogenes (L. monocytogenes) may grow to 3.6×10⁹ cfu in 24 hours assuming its growth rate as 2.5-fold per hour. Therefore, with 24-hour pre-enrichment step, PCR assay may detect as low as 1 cfu live L. monocytogenes with few false positive results due to dead organisms because very few food samples may be contaminated at such high level or ˜10⁹ cfu.

Recently, there is increasing desire and requirement from the food industry to shorten the total assay time or time to obtain final assay results to less than single work shift or 8 hours. Considering necessary time for sample preparation and detection assay, only 6 hours or less is allowed for pre-enrichment step to achieve pathogen detection within a single work shift. However, for example, 1 cfu live L. monocytogenes may grow to only 2.4×10² cfu in 6 hours assuming its growth rate as 2.5-fold per hour. Therefore, a PCR assay with 6-hour pre-enrichment step may face frequent false positive results due to dead organisms because some pasteurized food samples may be contaminated with dead organisms at low level or ˜10² cfu. Even using a very sensitive PCR assay, it is still very difficult to shorten the pre-enrichment step to 6 hours or less because of frequent false positive issues due to dead organisms and this is one of the disadvantages to employ PCR in food testing.

In order to avoid high false positive results in conventional PCR-based pathogen detection with pre-enrichment step, it would be desirable to employ an effective sample preparation method for live food pathogen detection especially for PCR detection.

While certain aspects of conventional technologies have been discussed to facilitate disclosure of the invention, Applicants in no way disclaim these technical aspects, and it is contemplated that the claimed invention may encompass or include one or more of the conventional technical aspects discussed herein.

### Definitions

Unless specifically defined herein, all technical and scientific terms have the same meaning as commonly understood by one of ordinary skill in the pertinent art. Also, all publications, patent application publications, and patents identified herein are incorporated by reference in their entirety.

As used herein, “selective agent” or “selective media” means an agent, or media containing one or more agents, that acts to inhibit growth of non-target or competing microorganisms in a culture.

As used herein, “configured to prevent clogging during filtration” means having an appropriate particle retention rate and thickness to trap target pathogen(s) efficiently, yet avoid filter clogging due to particles present in a sample undergoing filtration.

As used herein, “particle retention rate” of a filter is defined as a dimension of challenged particles which can be removed by the filter with 98% efficiency. Particle retention rate is similar to pore size in the case of membrane filter, but particle retention rate of depth filter is smaller than its pore size due to its thickness.

### SUMMARY

According to one aspect, the present invention provides a method to selectively detect a live pathogen in a sample containing a live and dead pathogens, without detecting the dead pathogen, the method comprising: (i) immobilizing at least a portion of the live and dead pathogens on a solid support with a physical barrier; (ii) incubating the solid support in a growth medium, where the live pathogen can multiply and the multiplied pathogen move from the solid support to a supernatant of the growth medium; and (iii) detecting the multiplied pathogen in the supernatant by a pathogen assay.

According to a further aspect, the present invention provides a method of selectively detecting a live pathogen in a sample comprising filtering the sample through a filter configured to attract the pathogen and having pores configured to prevent clogging during filtration, whereby the pathogen is collected in the filter, incubating the filter in a growth medium for a period of time sufficient for multiplication of the pathogen and diffusion of the multiplied pathogen to the growth medium; and detecting the presence of the multiplied pathogen in the growth medium as an indication of the pathogen by a pathogen assay.

According to yet another aspect, the present invention provides a method of detecting a pathogen in a particulate sample comprising filtering the particulate sample with a highly porous filter wherein a said filter configured to attract a pathogen and having pores configured to prevent clogging during filtration; incubating a said highly porous filter in elution solution comprising a growth medium, detergent, and chaotropic reagent or organic solvent to extract a said pathogen and/or its cellular component; and detecting the pathogen and/or its cellular component to identify the presence of the pathogen.

The present invention may address one or more of the problems and deficiencies of the prior art discussed above. However, it is contemplated that the invention may prove useful in addressing other problems and deficiencies, or provide benefits and advantages, in a number of technical areas. Therefore the claimed invention should not necessarily be construed as being limited to addressing any of the particular problems or deficiencies discussed herein.

### BRIEF DESCRIPTION OF THE DRAWINGS

These and other feature of this invention will now be described with reference to the drawings of preferred embodiments which are intended to illustrate and not to limit the invention.

FIG. 1 shows the steps of the pathogen detection assay procedure using a highly porous filter, porous spherical microbeads and pathogen extraction through culturing.

FIG. 2A is a schematic illustration of multiplication and transfer of live pathogens from a solid support to a growth medium.

FIG. 2B is a schematic illustration of retention of dead pathogens on a solid support.

FIG. 3A shows the speed of pathogen extraction from a highly porous filter through culturing after filtration of 50 mL 10% deli meat homogenate inoculated with 3.6 cfu (Δ) or 36 cfu (∘) L. monocytogenes. FIG. 3B shows the speed of pathogen extraction from a highly porous filter through culturing after filtration of 50 mL 10% deli meat homogenate inoculated with 6.5 cfu (Δ) or 65 cfu (∘) heat-injured L. monocytogenes.

FIG. 4A shows the results of pathogen detection assay of 25 g deli meat sample inoculated with 7.5 cfu or 75 cfu Listeria in a disclosed assay procedure using filtration of a food homogenate and pathogen extraction through culturing for 17 hours followed by immunochromatographic detection. FIG. 4B shows the results of pathogen detection assay of 25 g deli meat sample inoculated with 7.5 cfu or 75 cfu Listeria in a conventional assay procedure using incubation of food homogenates in 225 mL growth medium for 17 hours followed by immunochromatographic detection. FIG. 4C shows the results of pathogen detection assay of 25 g deli meat sample inoculated with 8.9 cfu or 89 cfu Listeria in 13 hours in total in a disclosed assay procedure using filtration of a food homogenate and pathogen extraction through culturing followed by immunochromatographic detection.

FIGS. 5A, 5B and 5C illustrate pathogen growth of L. monocytogenes, S. enterica and E. coli O157, respectively, over time.

FIGS. 6A, 6B and 6C illustrate the threshold cycles for assaying live and dead organism samples of L. monocytogenes, S. enterica and E. coli O157, respectively, in a disclosed assay procedure, as well as positive and negative assay controls.

FIGS. 7A, 7B and 7C illustrate the threshold cycles for assaying various food samples inoculated with each of L. monocytogenes, S. enterica and E. coli O157 at less than 1 cfu/mL or 1 cfu/mg levels in a disclosed assay procedure.

### DETAILED DESCRIPTION OF THE PREFERRED EMBODIMENTS

Further aspects, features and advantages of this invention will become apparent from the detailed description of the preferred embodiments which follow.

The present disclosure relates to a method that allows simultaneous detection of one or more live pathogens in multiple species in a large volume of particulate sample with low cost, a short hands-on time, and a short total assay time.

The methods and arrangements of certain optional aspects of the present invention may include a filter or methods having one or a combination of features and/or techniques of the description in WO 2009/018544, now U.S. patent application Ser. No. 12/671,675, now published as ______, the entire contents of which are incorporated by reference herein. In one embodiment, the disclosed method utilizes a highly porous filter with pathogen adsorption capability and high particulate holding capacity in order to efficiently concentrate a pathogen from a large volume particulate sample. Highly porous filters useful in embodiments of the disclosed method may attract target pathogens, preferably in multiple species, by electrostatic, hydrophilic, hydrophobic, physical, or biological interactions and also may be configured to prevent filter clogging by particulate samples. In certain embodiments, the filter deployed is a depth filter with a three-dimensional matrix that provides high particulate holding capacity and the ability to trap pathogens. The useful depth filter may be made of fibrous materials such as glass fiber and nitrocellulose fiber, and comprise of a single or multilayer filter with the same or different particle retention rate. The filter has appropriate particle retention rate and thickness to trap pathogens efficiently and to avoid filter clogging due to particles in the sample. In order to obtain high particle loading capacity, the filter may optionally comprise multiple layers of filter material with different particle retention rates arranged in decreasing order of their particle retention rates from the upstream side to the downstream side. For example, in order to trap a pathogen such as L. monocytogenes, which are 0.5-2 μm in length and 0.4-0.5 μm in diameter, in a depth filter, appropriate particle retention rate of depth filters may be 0.1 to 10 μm, preferably 0.7 to 2.4 μm. Also, the filter has sufficient mechanical strength to withstand vacuum forces or pressure applied during filtration.

In other embodiments, the filter is a depth filter that uses electropositive charges to attract pathogens, preferably in multiple species, as pathogen surfaces usually have a net negative charge on account of the lipopolysaccharides, teichoic acids, and surface proteins contained therein. The pathogen may be immobilized by the electropositive charges on the filter rather than by the filter matrix, thus making it possible to further increase filter porosity and obtain a higher particulate-holding capacity to avoid filter clogging more efficiently. The electropositive charges may be provided by surface coating of filter matrix with cationic molecules or incorporating electropositive colloids, particles or fibers made of electropositive materials. The examples of the electropositive materials are metal hydroxides and metal oxides, such as zirconium hydroxide, titanium hydroxide, hafnium oxide, iron oxide, titanium oxide, aluminum oxide, and hydroxyapatite. Preferably, the isoelectric point of the metal hydroxides or metal oxides may be higher than the pH values of the sample and detection reagent. There are a few rare instances of positively-charged bacteria, such as Stenotrophomonas maltophilia. Such microorganisms can be more readily retained by filters with an electronegative charge, which can be prepared in a manner similar to the electropositive filters described above, using electronegative charges rather than electropositive ones.

In other embodiments, the filter comprises pathogen recognition agents such as antibodies, antigens, proteins, nucleic acids, carbohydrates, aptamers, or bacteriophages. These pathogen recognition agents can recognize pathogens selectively or non-selectively vis-à-vis other microorganisms found in the sample and can be immobilized on the filter matrix by chemical binding, physical binding, or other standard immobilization methods. In certain embodiments, the pathogen recognition agent is a toll-like receptor (TLR), which recognizes structurally conserved molecules present on the surface of various microorganisms. TLR2 can recognize Gram-positive peptidoglycan and lipoteichoic acid, and TLR4 can recognize lipopolysaccharides on Gram-negative bacteria.

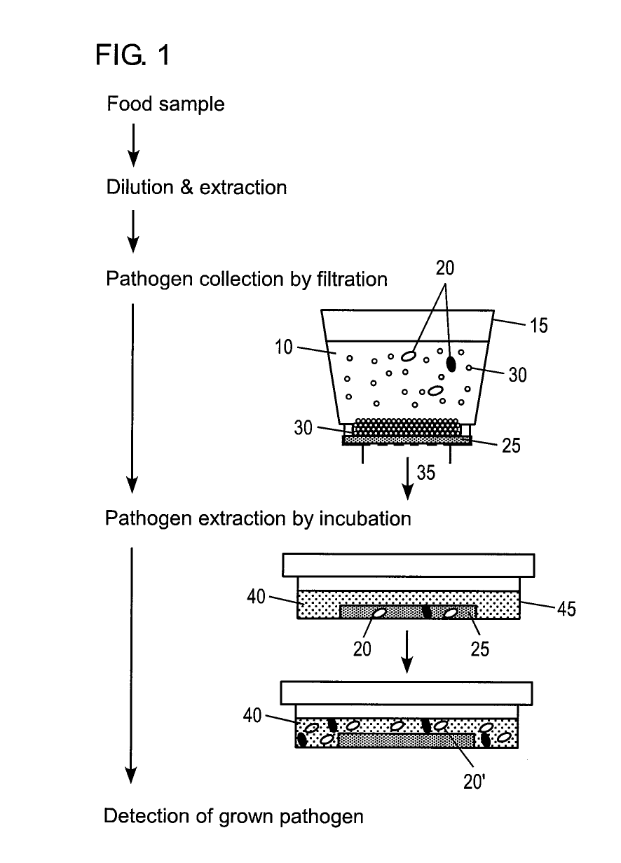

In addition to the filter, the disclosure relates to the use of a porous spherical microbeads filter aid to prevent filter clogging by particulates in the sample to be tested (FIG. 1). Useful microbeads may be spherically, spheroidally or ellipsoidally shaped porous microbeads with a small size distribution. The microbeads may take the closest packed structure such as cubic closest packed structure and hexagonal closest packed structure or close structure to that. The microbeads may typically have a diameter of 1 to 1000 μm, preferably 15 to 600 μm, and more preferably 50 to 300 μm, in order for the pathogen to be tested to pass among the microbeads during sample filtration. The useful microbeads have appropriate specific gravity to be suspended but not to float in water, buffer, growth medium or sample solution. For example, the microbeads can have a specific gravity of between 1.0 and 1.5 in a wet condition, preferably between 1.0 and 1.3 in wet condition. The microbeads may typically have pores that are smaller than a target pathogen, thereby preventing the pathogen from being trapped inside the pores of the porous spherical microbeads during sample filtration. Preferably, the microbeads may have an inert surface, preferably hydrophilic surface, and have low non-specific binding to biomolecules and microorganisms, including proteins, nucleic acids, carbohydrates, bacteria, viruses, and organisms. In one optional embodiment, the microbeads are made of a suitable material, such as a cross-linked polymer such as polymethacrylate and dextran. Optionally, the microbeads can remove materials that may inhibit the downstream detection reaction on account of their porous structure.

Porous spherical microbeads may be used to aid in the filtration of a large volume particulate sample at least in one of the following ways, or combinations thereof. A filter aid can be placed as a homogeneous or graded layer of porous spherical microbeads on the upstream surface of a highly porous filter with pathogen adsorption capability before sample filtration. Alternatively, a filter aid can be added to the particulate sample before filtration and form a layer of porous spherical microbeads during sample filtration, thereby providing new layers of the microbeads continuously and further improving filtration. A mesh support may be placed between the highly porous filter and the filter aid layer in order to remove the filter aid layer easily after sample filtration. Optionally, the porous microbead are pre-incubated or suspended in a solution containing a blocking reagent such as a peptide or protein before use in order to minimize pathogen binding to the microbead surfaces.

The pathogen to be detected may pass among the microbeads and are not typically trapped on the surfaces or in the pores of the microbeads. On the other hand, particles in the sample, that typically clog a filter without a filter aid layer, may be trapped on the microbeads surface and in the gaps among the porous spherical microbeads. Because of the porous structure of the microbeads, the sample streams during filtration not only pass among the beads but also penetrate the microbeads themselves, therefore the sample particles tend to be trapped on the beads surface rather than in the gaps among the beads, and thereby the porous spherical filter aid layer can provide high particulate holding capacity and prevent filter clogging during filtration of large volume particulate samples. After complete filtration of the sample, the filter aid layer can be disrupted and the microbeads can be suspended in a wash solution, and the wash solution can be filtered to collect the pathogen that may be trapped in the filter aid layer during the initial filtration of the particulate sample, thereby improving pathogen immobilization yield in the filter. This wash process can be repeated several times to maximize the pathogen recovery yield in the filter. The wash solution is typically a buffer solution or growth medium that is not harmful to the pathogen.

In certain embodiments, a growth medium may be used. The growth medium generally includes one or more of a carbon source, nitrogen source, amino acids, and various salts for pathogen growth. In some embodiments, the growth medium may be a non-selective or selective media to support simultaneous growth of pathogens in multiple species such as L. monocytogenes, Salmonella species and Escherichia coli (E. coli) O157 and rapid resuscitation of pathogens if injured by sample conditions such as heat, cold, acid, alkali, refrigeration, freeze, pressure or vacuum. Some growth media such as universal pre-enrichment broth (UPB), No. 17 and Salmonella, E. coli, and L. monocytogenes enrichment media (SEL) as disclosed in U.S. Pat. No. 5,145,786, U.S. Patent Application Publication No. 2008/0014578 and Appl. Environ. Microbiol. 2008, 74, 4853-4866, respectively, (all of which are hereby incorporated by reference in their entirety) may be especially useful in this invention because those media were developed to support simultaneous growth of pathogens in multiple species such as L. monocytogenes, Salmonella species and E. coli O157 from food samples. UPB is highly buffered and low in carbohydrates to prevent rapid pH decreases due to growth of competing microorganisms in culture. Therefore, UPB can support simultaneous enrichment of even injured pathogens. No. 17 has a similar medium composition to UPB. SEL was developed based on buffered Listeria enrichment broth (BLEB) which is a Listeria selective growth medium, by reducing concentrations of antibiotics to support growth of Salmonella species and E. coli O157 in addition to that of L. monocytogenes. Other non-selective growth media such as Brain Heart Infusion Broth (BHI), Nutrient Broth (NB) and Tryptic Soy Broth (TSB) may be useful although those growth media are known to show Jameson effect, i.e. a phenomenon that high total microorganism concentration in a culture suppresses growth of all microorganisms. Alternatively, the growth medium can be a selective growth medium which includes one or more selective agents such as antibiotic against competing microorganisms. Selective media with high concentrations of selective agents are nevertheless undesirable because selective agents at high concentrations can prevent resuscitation of injured pathogens and decrease growth rates of all pathogens in a culture. Optionally, the growth medium may contain compounds such as L-cysteine and Oxyrase (Oxyrase Inc., Mansfield, Ohio) to accelerate the growth of specific pathogens and/or resuscitation of injured pathogens by reducing oxygen concentration in the growth medium.

In certain optional embodiments, detection of one or more pathogens in a large volume of particulate sample comprises (FIG. 1):
(1) Filter a large volume of particulate sample 10 in a filter housing 15 that may contain the pathogens 20 in multiple species through a layered filter comprising a highly porous filter 25 and a porous microbeads 30 filter aid, thereby collecting the target pathogens 20 in the filter. Optionally, filtration is assisted by a vacuum 35.
(2) If necessary, wash the filter 25 and the porous microbeads 30 filter aid in order to maximize recovery of the pathogens in the filter and to remove any potential inhibitors of pathogen growth or pathogen detection assay.
(3) Collect the filter 25 and incubate the filter 25 in a growth medium 40 for a period of time sufficient for resuscitation of the pathogens 20 if injured, multiplication of the pathogens 20, and diffusion of the multiplied pathogens 20 into the growth medium 40 such that the pathogens 20 will be detectable.
(4) Detect the multiplied pathogen 20′ in the growth medium 40 using a pathogen detection assay.

Steps 1 and 2 can be completed within 5 to 30 minutes in the case of typical food samples, for example, 250 mL 10% (w/v) deli meat and hot dog homogenates. Necessary time for Step 3 depends on pathogen concentration in samples, doubling time of the pathogen and the sensitivity of pathogen detection assay in Step 4. For example, if real-time PCR (1 to 100 cfu sensitivity) is used in Step 4, Step 3 may be completed within 1 hour to 8 hours. If a less sensitive assay such as immunochromatography (10⁵ to 10⁶ cfu sensitivity) is used, Step 3 may take longer until the pathogen concentration reaches assay sensitivity. Step 4 can be completed within a few minutes to a few hours depending on assay selected.

Optionally, in order to resuscitate and detect an injured pathogen, the particulate sample may be prepared and incubated in a growth medium or solution allowing resuscitation of the injured pathogen before sample filtration.

The filter incubation can be done in a container such as Petri dish and centrifuge tube with or without agitating, shaking, rotating or mixing in order to promote the multiplication of the pathogen and/or diffusion of the multiplied pathogen from the filter. The filter incubation can be done in continuous or non-continuous flow of the growth medium and the growth medium may be circulated if continuous flow is used. The volume of the growth medium may be preferred to be as low as possible in order to extract the pathogen immobilized in the filter into small volume, thereby the pathogen concentration in the growth medium will be higher and the pathogen can be detected earlier. The volume of the growth medium may be less than 50 mL, less than 10 mL, or less than 5 mL.

Target pathogens in multiple species such as L. monocytogenes, Salmonella species and E. coli O157 can be assayed simultaneously by using a non-selective growth medium such as UPB, No. 17, BHI, NB and TSB to support simultaneous growth of these pathogens. Generally, non-target microorganisms in samples may interfere with isolation of target pathogens by multiplying more rapidly, causing exhaustion of nutrient and energy sources in the growth medium, and decrease in the pH of the growth medium, therefore growth of the target pathogens may be inhibited before reaching the detectable level (Jameson effect). However, in the disclosed pathogen detection assay, filtration can reduce the sample volume significantly and remove food debris, which may inhibit the growth of the pathogens, therefore it is possible for the target pathogens to grow to the detectable level even in a non-selective growth medium before reaching the decline phase of organism growth. Alternatively, selective growth medium supporting simultaneous growth of multiple target pathogens, but inhibiting the growth of other non-target microorganisms can be used. One example of such selective growth media is SEL broth for simultaneous growth of L. monocytogenes, Salmonella species and E. coli O157. Single species of target pathogen can be tested as well as pathogens in multiple species using non-selective or selective growth medium supporting growth of the pathogen. However, it is more preferable to conduct assays for pathogens in multiple species simultaneously in order to increase assay throughput and cost performance.

Incubation of the highly porous filter in a small volume of growth medium allows the pathogens immobilized in the filter to multiply and the multiplied pathogens come out into the solution phase of the growth medium along with the incubation, therefore pathogen detection can be done by assaying the presence of the multiplied pathogens in the growth medium. The pathogen or the cellular components of the pathogen such as genomic DNA, ribosomal RNA, transfer RNA, messenger RNA, or protein, which indicate the presence of the specific pathogen, can be extracted from the supernatant and used for detection. Extraction of those cellular components can be done using standard molecular biology techniques or commercial products. Useful pathogen detection assay is chromogenic agar plate, polymerase chain reaction (PCR), reverse transcription polymerase chain reaction (RT-PCR), nucleic acid sequence based amplification (NASBA), loop-mediated isothermal amplification (LAMP), any other nucleic acid amplification, enzyme linked immunosorbent assay (ELISA), immunochromatography, biosensor, or nucleic acid probe.

When multiple target pathogens are assayed, the supernatant of the growth medium after filter incubation or the extracted cellular components can be split and used for detection of each pathogen individually. Alternatively, the supernatant of the growth medium after filter incubation or the extracted cellular components can be tested by multiplex pathogen detection assay such as multiplex PCR, multiplex ELISA, DNA microarray, protein microarray and Luminex assay to detect the multiple target pathogens simultaneously.

Alternatively, the pathogen or cellular components of the pathogen such as genomic DNA, ribosomal RNA, transfer RNA, messenger RNA, or protein, which indicate the presence of the specific pathogen, can be extracted from the filter by use of a growth medium, detergent, chaotropic reagent, organic solvent, or electrophoresis with or without breaking the filter structure, and detected using the conventional pathogen detection methods as described above. A useful detergent or chaotropic reagent may be Tween-20, CHAPS, Triton X series, NP40, sodium dodecyl sulfate or guanidinium chloride. Enzymes such as lysozyme and proteinase K or organic solvents such as DMSO may be included to enhance the collection yield of the pathogens and/or their cellular components. Any physical methods such as shaking, heating and homogenizing of the filter may be combined to enhance the collection efficiency if necessary.

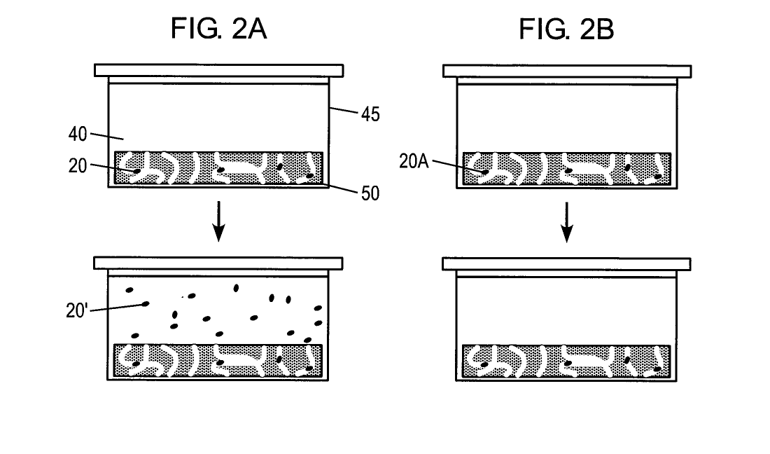

Additional embodiments of the present invention regarding a method of selective live pathogen detection are illustrated in FIGS. 2A-2B. As illustrated therein, live/dead pathogens 20/20A may be immobilized permanently or semi-permanently in a solid support 50 via electrostatic interaction, hydrogen bonding, hydrophobic interaction or antibody-antigen interaction, etc., however, live pathogens can multiply during incubation of the solid support 50 in a growth medium 40 as described above and the multiplied pathogens 20 can come out to the growth medium phase before being immobilized again in the solid support. Even if the multiplied pathogens are immobilized in the solid support during incubation, their descendants can still come out to the growth medium. On the other hand, the dead pathogen does not multiply, therefore no dead pathogen will be observed in the growth medium. Therefore, incubation of the solid support allows selective release of the multiplied live pathogens into the growth medium and keeping the dead pathogen in the solid support. Additionally, the solid support may have a physical barrier such as three-dimensional matrix, maze-like structure, mesh, pores, etc. to avoid the immobilized dead pathogens from being released into the growth medium phase during incubation because this could become false positive results by the following pathogen detection such as PCR. One example of the solid support is a highly porous filter 25 such as a filter with a three-dimensional matrix as described above. By assaying the presence of the multiplied pathogens in the growth medium after incubation, selective detection of live pathogen can be achieved without having false positive results due to the dead pathogen in a sample. Especially when PCR assay or other nucleic acid amplification technology, which is rapid and sensitive, but may not distinguish live and dead pathogens, is used for detection of the multiplied pathogens, the disclosed method allows rapid, sensitive and live pathogen detection.

Optionally, the solid support 50 may be made of or coated with repellent material for pathogens (for example, toxic but not life threatening), therefore during incubation, the multiplied pathogens may move to growth medium phase through chemotaxis (organisms move from toxic area to non-toxic area).

Examples of pathogens that may be detected include pathogenic bacteria and other microorganisms infectious or harmful to humans, animals, plants, the environment, and/or industry. Examples of pathogenic bacteria include, but are not limited to, Escherichia, Salmonella, Listeria, Campylobacter, Shigella, Brucella, Helicobactor, Mycobacterium, Streptococcus, and Pseudomonas. Pathogenic virus can be detected in combination with a conventional pathogen detection method as disclosed herein. Examples of pathogenic virus families include, but are not limited to, Adenoviridae, Picornaviridae, Herpesviridae, Hepadnaviridae, Flaviviridae, Retroviridae, Orthomyxoviridae, Paramyxoviridae, Papovaviridae, Rhabdoviridae, and Togaviridae. In addition, the disclosed filtration system is useful for detecting both pathogenic and non-pathogenic microorganisms in large volumes of particulate samples.

Examples of particulate samples that can be tested include, but are not limited to, food samples, homogenates of food samples, wash solutions of food samples, drinking water, ocean/river water, environment water, mud and soil. Additionally, swabs, sponges and towels wiping a variety of environment surfaces can be tested as well. In addition, the disclosed filtration system is useful to test other large volume particulate samples including human body fluids, urine, blood and manufacturing water/solution. Samples, especially solid forms, can be diluted, homogenized and/or pre-filtered with a mesh filter having 50-1000 μm pores before sample filtration for more efficient filtration of the sample.

### EXAMPLE 1

50 mL 10% deli meat homogenates inoculated with various doses of L. monocytogenes were tested by a disclosed pathogen detection assay.

L. monocytogenes was cultured overnight in BHI (brain-heart infusion) broth at 37° C. and the pathogen concentration was estimated by colony counting. Heat-injured L. monocytogenes was prepared by heating the freshly cultured L. monocytogenes at 50° C. for 10 min in BHI broth. 10% deli meat homogenates were prepared by homogenizing 25 g deli meat in 225 mL PBS by a stomacher (Seaward, UK) at 230 rpm for 2 min. 50 mL 10% deli meat homogenates were inoculated with various doses of L. monocytogenes before conducting a pathogen detection assay.

A pathogen detection assay was conducted as follows: (1) 50 mL 10% deli meat homogenate inoculated with L. monocytogenes was filtered with GMF150 1 μm filter (Whatman, NJ) by vacuum filtration, (2) the filter was transferred to a sterile container , (3) the filter was incubated in 5 mL of half fraser broth at 30° C. for 4, 6 or 8 hours, and (4) 100 μL of the supernatant was used for a pathogen detection assay using RAPID L'mono agar (Bio-Rad, CA).

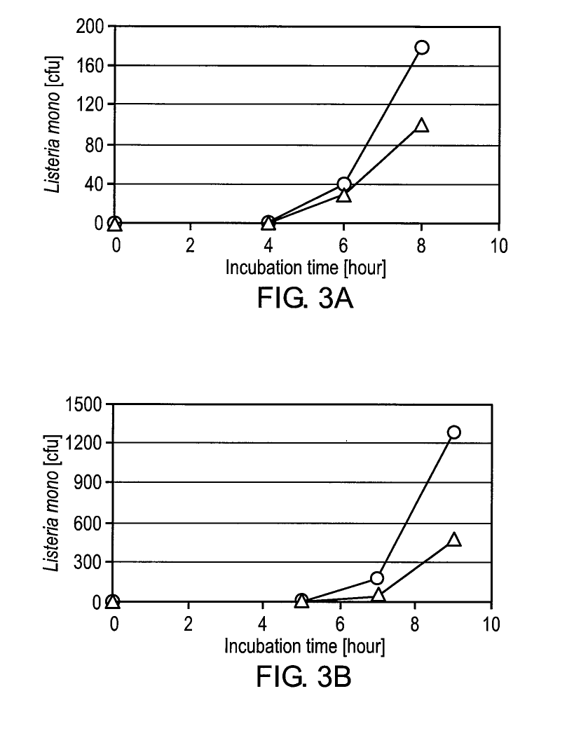

50 mL 10% deli meat homogenates inoculated with 3.6 or 36 cfu L. monocytogenes were tested in duplicate. The average number of the observed L. monocytogenes colonies on RAPID L'mono agar at different incubation time is shown in FIG. 3A. This data indicate that L. monocytogenes immobilized in GMF150 1 μm were successfully extracted into the supernatant through culturing and 6-hour incubation was enough for the detection by RAPID L'mono agar.

50 mL 10% deli meat homogenates inoculated with 6.5 or 65 cfu heat-injured L. monocytogenes were also tested. The average number of the observed L. monocytogenes colonies on RAPID L'mono agar at different incubation time is shown in FIG. 3B. This data indicate that heat-injured L. monocytogenes immobilized in GMF150 1 μm were successfully resuscitated and extracted into the growth medium through culturing and 7-hour incubation was enough for the detection by RAPID L'mono agar.

### EXAMPLE 2

25 g deli meat samples inoculated with various doses of Listeria were tested by a disclosed pathogen detection assay.

Listeria innocua was cultured overnight in BHI broth at 37° C. and the Listeria concentration was estimated by colony counting. 25 g deli meat samples were artificially inoculated on the surface with various doses of Listeria and dried at room temperature at least 30 min.

A pathogen detection assay was conducted as follows: (1) 25 g deli meat sample inoculated with Listeria was homogenized in 225 mL PBS by a stomacher (Seaward, UK) at 230 rpm for 3 min, (2) the food homogenate was filtered with GMF150 1 μm filter (Whatman, NJ) with the assistance of 2 g porous spherical microbeads (cross-linked polymethacrylate, EG50OH, Hitachi Chemical, Japan) by vacuum filtration, (2) the filter was transferred to a sterile container and incubated in 5 mL of LESS medium (Neogen, MI) at 30° C. for 17 hours, and (3) 135 μL of the supernatant was used for an immunochromatographic detection assay using Reveal for Listeria (Neogen, MI).

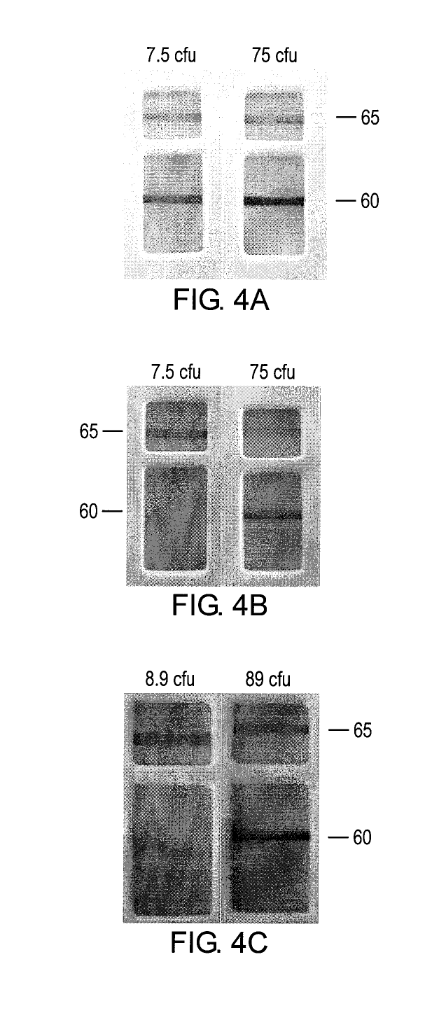

25 g deli meat sample inoculated with 7.5 or 75 cfu were tested (FIG. 4A). With 17-hour incubation, very strong positive signals in the test windows 60 when compared with the control windows 65 were obtained for both 7.5 and 75 cfu samples, indicating the presence of Listeria.

For reference, 25 g deli meat sample inoculated with 7.5 or 75 cfu was tested in a conventional assay protocol, where (1) a food sample was incubated in 225 mL LESS medium at 30° C. for 17 hours and (2) 135 μL of the supernatant was used for a pathogen detection assay using Reveal for Listeria, and only weak positive signals were obtained (FIG. 4B). The strong positive signals similar to FIG. 4A were obtained after 24-hour incubation. These data suggest that the disclosed pathogen detection assay can detect as low as 7.5 cfu Listeria in 25 g deli meat sample at least 7 hours earlier than the conventional assay protocol.

### EXAMPLE 3

25 g deli meat samples inoculated with various doses of Listeria were tested by a pathogen detection assay.

A pathogen detection assay was conducted as follows: (1) 25 g deli meat sample inoculated with Listeria was homogenized in 225 mL PBS by a stomacher (Seaward, UK) at 230 rpm for 3 min, (2) the food homogenate was filtered with GMF150 1 μm filter (Whatman, NJ) with the assistance of 2 g porous spherical microbeads (cross-linked polymethacrylate, EG50OH, Hitachi Chemical, Japan) by vacuum filtration, (3) the filter was transferred to a sterile container and incubated in 5 mL of LESS medium (Neogen, MI) at 30° C. for 12 hours, (4) the grown Listeria in 2 mL of the supernatant was concentrated into 150 μL volume by centrifugation at 10,000 rpm for 5 min, and (5) 135 μL of the concentrated sample was used for an immunochromatographic detection assay using Reveal for Listeria (Neogen, MI).

25 g deli meat sample inoculated with 8.9 or 89 cfu were tested with the above protocol (FIG. 4C). Within 13 hours in total, as low as 8.9 cfu Listeria in 25 g deli meat sample was successfully detected, meaning that assay results can be obtained at least 11 hour earlier than a conventional assay using the same detection method.

### EXAMPLE 4

The samples used here are 10 mL of 10% deli meat homogenates inoculated with various concentrations of food pathogens (L. monocytogenes, S. enterica and E. coli O157). A depth filter made of glass fiber was used as a solid support with a physical barrier. Pathogen immobilization was done by vacuum filtration of the food homogenates. Incubation of the solid support was done in 5 mL universal pre-enrichment broth (UPB) at 37° C. up to 8 hours. Pathogen detection was conducted by chromogenic agar or real-time PCR using a fraction of the growth medium.

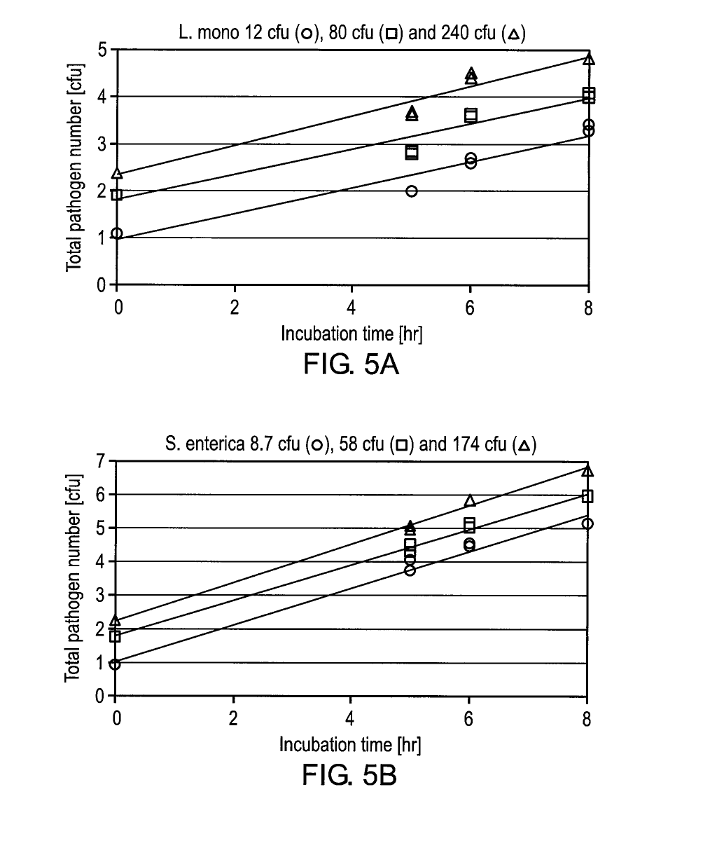

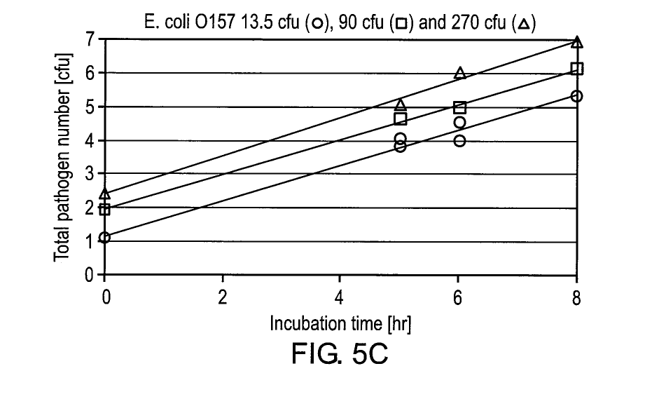

As shown in FIGS. 5A-5C, chromogenic agar data, the pathogens were detected in the growth medium successfully after 5- to 8-hour incubation of the solid support. Real-time PCR detection also successfully detected L. monocytogenes, S. enterica and E. coli O157 in the growth medium in separate experiments with 6- and 8-hour incubation.

### EXAMPLE 5

L. monocytogenes, S. enterica and E. coli O157 were cultured overnight at 37° C. in 5 mL BHI broth. Organism concentrations were determined by plate counting. Dead organisms of L. monocytogenes, S. enterica and E. coli O157 were prepared by heating the organisms at 60° C. for 30 min and their sterility was confirmed by plating on BHI agar plate.

First, in order to confirm that dead organisms still maintain their genomic DNA, genomic DNA was prepared from 10⁵ cfu live or dead organisms and analyzed by real-time PCR as follows. Threshold cycles of live and dead organisms were similar, therefore it was concluded that heat treatment does not deteriorate genomic DNA or organism structure of L. monocytogenes, S. enterica and E. coli O157.

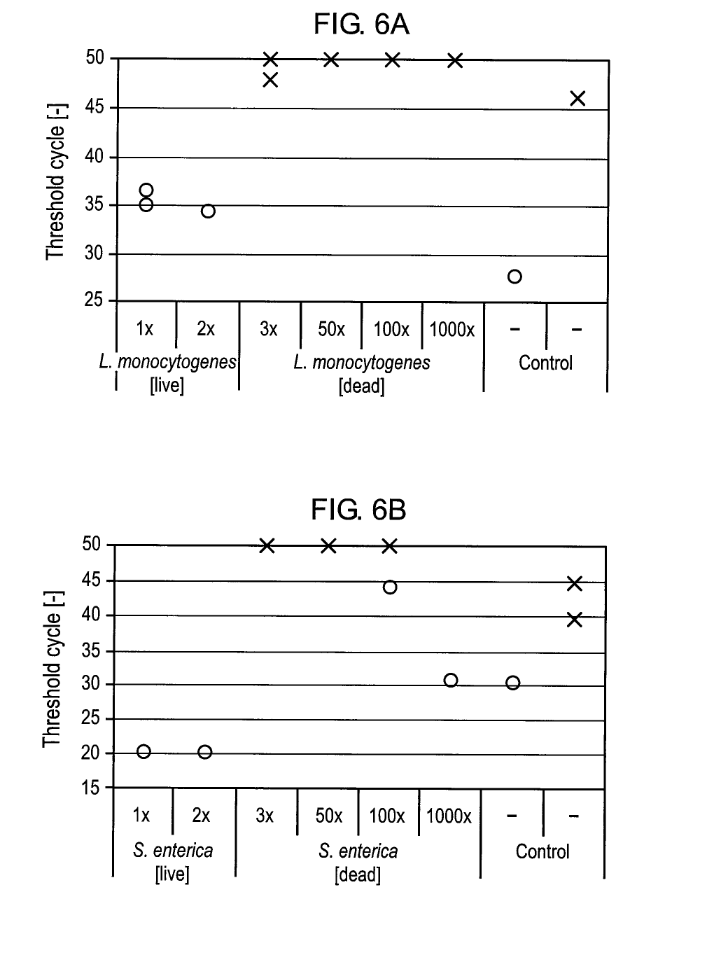

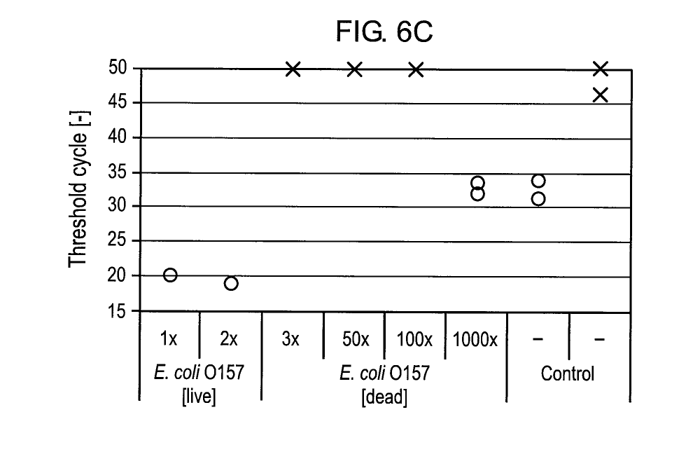

Second, in order to confirm that the disclosed assay procedure can eliminate the false positive of PCR due to dead organisms and can distinguish small amount of live organisms in the presence of excess dead organisms, the following experiment was conducted. 10 mL BHI broth samples were inoculated with various amount of live and dead organisms: 13 (1×), 130 (10×), 1,300 (100×) and 13,000 cfu (1000×) L. monocytogenes, 2.7 (1×), 27 (10×), 270 (100×) and 2,700 cfu (1000×) S. enterica and 8.0 (1×), 80 (10×), 800 (100×) and 8,000 (1000×) cfu E. coli O157. These samples were filtered with a glass fiber depth filter, GMF150 1 μm (Whatman, NJ) by vacuum aspiration. The filters were placed in a container and incubated in 5 mL universal pre-enrichment broth at 37° C. for 6 hours. 2 mL supernatants were removed and genomic DNA was prepared by Quick-gDNA Microprep kit (Zymo Research, CA) and quantified by real-time PCR. As shown in FIGS. 6A, 6B and 6C respectively, live L. monocytogenes, S. enterica, and E. coli O157 pathogens were successfully detected by real time PCR with low threshold cycles, while dead pathogens were not detected or detected only with high threshold cycles. Open circles indicate threshold cycles with appropriate melting curves and crosses indicate threshold cycles without appropriate melting curves (50 for undetected samples). Positive and negative controls are purified genomic DNA and water, respectively.

### EXAMPLE 6

Various food samples (100 mL whole milk, 25 mL orange juice or 25 g deli meat) were inoculated with L. monocytogenes, S. enterica and E. coli O157 at less than 1 cfu/mL or 1 cfu/mg levels.

A pathogen detection assay was conducted as follows: (1) 100 mL whole milk and 25 mL orange juice samples were diluted in 100 mL and 225 mL PBS, respectively, 25 g deli meat samples were homogenized in 225 mL PBS by a stomacher (Seaward, UK) at 200 rpm for 30 sec, (2) each food homogenate was filtered with GMF150 1 μm filter (Whatman, NJ) with the assistance of 0.5 g to 1 g porous spherical microbeads (cross-linked polymethacrylate, EG50OH, Hitachi Chemical, Japan) by vacuum filtration, (3) the filter was incubated in 5 mL UPB at 37° C. for 6 hours, (4) genomic DNA was extracted from the supernatant by Quick-gDNA Microprep kit (Zymo Research, CA) and detected by real-time PCR.

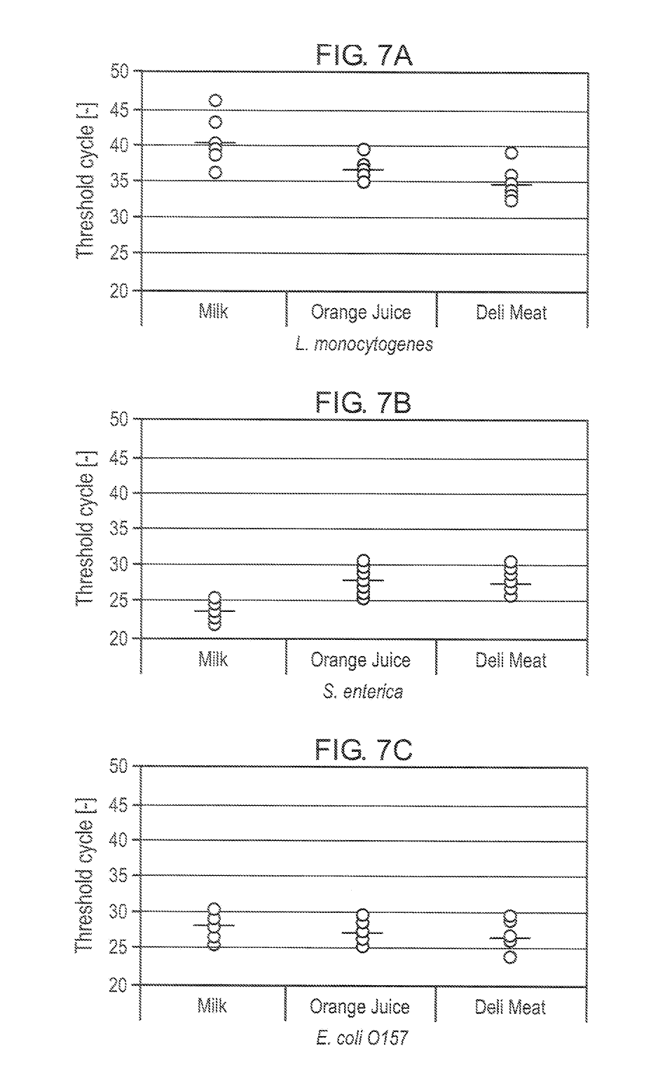

As shown in FIGS. 7A, 7B and 7C, L. monocytogenes, S. enterica and E. coli O157 at less than 1 cfu/mL or 1 cfu/mg levels in various food samples were successfully detected within 8 hours in total. Open circles indicate threshold cycles of each food sample and black horizontal bars indicate the average of the threshold cycles. Table 1 below summarizes the assay results with assay accuracies.

TABLE 1

| | | | | | positive/ | |
|---|---|---|---|---|---|---|
| Sample | | | | | sample | % |
| L. mono- | 0.36 cfu/mL | 100 | mL | Milk | 7/9 | 78% |
| cytogenes | 0.43 cfu/mL | 25 | mL | Orange Juice | 7/9 | 78% |
| | 0.30 cfu/mg | 25 | g | Deli Meat | 10/10 | 100% |
| S. enterica | 0.32 cfu/mL | 100 | mL | Milk | 9/9 | 100% |
| | 0.20 cfu/mL | 25 | mL | Orange Juice | 9/9 | 100% |
| | 0.30 cfu/mg | 25 | g | Deli Meat | 10/10 | 100% |
| E. coli O157 | 0.34 cfu/mL | 100 | mL | Milk | 8/9 | 89% |
| | 0.76 cfu/mL | 25 | mL | Orange Juice | 9/9 | 100% |
| | 0.19 cfu/mg | 25 | g | Deli Meat | 8/10 | 80% |

### EXAMPLE 7

25 g various samples (deli meat, ground beef, hot dog, romaine lettuce, milk and orange juice) were inoculated with L. monocytogenes, S. enterica and E. coli O157 at 1 to 10 cfu per 25 g levels.

A pathogen detection assay was conducted as follows: (1) a 25 g food sample was homogenized or diluted in 225 mL UPB and incubated at 37° C. for 6 hours, (2) the food homogenate was filtered with GMF150 1 μm filter (Whatman, NJ) with the assistance of 0.5 g to 1.0 g porous spherical microbeads (cross-linked polymethacrylate, EG50OH, Hitachi Chemical, Japan) by vacuum filtration, (3) the immobilized pathogen in the filter was extracted with 5 mL Elution Buffer (10 mM Tris-HCl, pH 8.0, 1 mM EDTA and 0.5% (v/v) Tween-20), (4) genomic DNA was extracted and detected by real-time PCR using iQ check Listeria II, Salmonella II and E. coli O157 kits (Bio-rad, CA).

As summarized in Table 2 below, L. monocytogenes, S. enterica and E. coli O157 in 25 g various food samples were successfully detected by real-time PCR within 8 hours with 100% assay accuracy.

TABLE 2

| | | | | Ro- | | |
|---|---|---|---|---|---|---|
| | Deli | Ground | | maine | | Orange |
| Pathogens | meat | beef | Hot dog | lettuce | Milk | juice |
| L. | 100% | 100% | 100% | 100% | 100% | 100% |
| monocytogenes | (n = 14) | (n = 12) | (n = 8) | (n = 8) | (n = 8) | (n = 8) |
| <10 cfu/25 | | | | | | |
| gram | | | | | | |
| S. enterica | 100% | 100% | 100% | 100% | 100% | 100% |
| <10 cfu/25 | (n = 14) | (n = 12) | (n = 8) | (n = 8) | (n = 8) | (n = 8) |
| gram | | | | | | |
| E. coli O157 | 100% | 100% | 100% | 100% | 100% | 100% |
| <10 cfu/25 | (n = 14) | (n = 12) | (n = 8) | (n = 8) | (n = 8) | (n = 8) |
| gram | | | | | | |

Any numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention.

1. A method to selectively detect a live pathogen in a sample containing a live and dead pathogens, without detecting the dead pathogen, the method comprising:
immobilizing at least a portion of the live and dead pathogens on a solid support with a physical barrier;
incubating the solid support in a growth medium, where the live pathogen can multiply and the multiplied pathogen move from the solid support to a supernatant of the growth medium; and
detecting the multiplied pathogen in the supernatant by a pathogen assay.

2. The method of claim 1, wherein the solid support is a filter configured to attract the pathogen to be detected and having pores configured to prevent clogging during filtration.

3. The method of claim 2, wherein the filter is a depth filter comprising a fibrous material, the fibrous material comprising cellulose fibers and glass microfibers.

4. The method of claim 2, wherein the filter is configured to attract the pathogen by an interaction selected from the group consisting of electrostatic, hydrophilic, hydrophobic, physical, and biological interactions.

5. The method of claim 2, wherein the filter further comprises a homogeneous or graded layer of porous spherical microbeads.

6. The method of claim 1, wherein the live pathogen comprises at least two species.

7. The method of claim 6, wherein the growth medium supports simultaneous growth of at least two species of the live pathogens.

8. The method of claim 6, wherein the pathogen assay detects the multiplied pathogens in at least two species separately or simultaneously.

9. The method of claim 1, wherein the growth medium is a non-selective growth medium.

10. A method of claim 1, wherein the pathogen assay comprises an agar plate, chromogenic agar plate, enzyme-linked immunosorbent assay (ELISA), immunochromatography, polymerase chain reaction (PCR), reverse transcription PCR (RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system.

11. The method of claim 1, wherein the method is completed in about 1 to about 24 hours.

12. The method of claim 11, wherein the sample contained less than 1,000 cfu of the pathogen to be detected.

13. A method of selectively detecting a live pathogen in a sample comprising:
filtering the sample through a filter configured to attract the pathogen and having pores configured to prevent clogging during filtration, whereby the pathogen is collected in the filter,
incubating the filter in a growth medium for a period of time sufficient for multiplication of the pathogen and diffusion of the multiplied pathogen to the growth medium; and
detecting the presence of the multiplied pathogen in the growth medium as an indication of the pathogen by a pathogen assay.

14. The method of claim 13, further comprising:
washing the filter after the filtering step for a period of time sufficient to remove substances that inhibit the detection or growth of the pathogen.

15. The method of claim 13, wherein the filter is a depth filter comprising a fibrous material comprising cellulose fibers and glass microfibers.

16. The method of claim 13, wherein the filter is configured to attract the pathogen to be detected by an interaction selected from the group consisting of electrostatic, hydrophilic, hydrophobic, physical, and biological interactions.

17. The method of claim 13, wherein the filter further comprises a homogeneous or graded layer of porous spherical microbeads.

18. The method of claim 13, wherein the live pathogen comprises at least two species.

19. The method of claim 18, wherein the growth medium supports simultaneous growth of at least two species of live pathogens.

20. The method of claim 18, wherein the pathogen assay detects the multiplied pathogens in at least two species separately or simultaneously.

21. The method of claim 13, wherein the growth medium is a non-selective growth medium.

22. The method of claim 13, wherein the pathogen assay comprises an agar plate, chromogenic agar plate, enzyme-linked immunosorbent assay (ELISA), immunochromatography, polymerase chain reaction (PCR), reverse transcription PCR (RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system.

23. The method of claim 13, wherein the method is completed in about 1 to about 24 hours.

24. The method of claim 23, wherein the sample contained less than 1,000 cfu of the pathogen to be detected.

25. A method of detecting a pathogen in a particulate sample comprising:
filtering the particulate sample with a highly porous filter wherein a said filter configured to attract a pathogen and having pores configured to prevent clogging during filtration;
incubating a said highly porous filter in a small volume of elution solution comprising a growth medium, detergent, chaotropic reagent or organic solvent to extract a said pathogen and/or its cellular component; and
detecting the pathogen and/or its cellular component to identify the presence of the pathogen.

26. The method of claim 25, wherein the solid support is a filter configured to attract the pathogen to be detected and having pores configured to prevent clogging during filtration.

27. The method of claim 26, wherein the filter is a depth filter comprising a fibrous material, the fibrous material comprising cellulose fibers and glass microfibers.

28. The method of claim 26, wherein the filter is configured to attract the pathogen by an interaction selected from the group consisting of electrostatic, hydrophilic, hydrophobic, physical, and biological interactions.

29. The method of claim 26, wherein the filter further comprises a homogeneous or graded layer of porous spherical microbeads.

30. The method of claim 25, wherein the pathogen comprises at least two species.

31. The method of claim 30, wherein the pathogen assay detects the multiplied pathogens in at least two species separately or simultaneously.

32. A method of claim 30, wherein the pathogen assay comprises a agar plate, chromogenic agar plate, enzyme-linked immunosorbent assay (ELISA), immunochromatography, polymerase chain reaction (PCR), reverse transcription PCR (RT-PCR), real-time PCR, real-time RT-PCR, nucleic acid sequence based amplification (NASBA), loop-mediated isothermal amplification (LAMP), isothermal nucleic acid amplification, nucleic acid probe, biosensor, multiplex PCR, multiplex real-time PCR, DNA microarray, protein microarray or Luminex system.

33. The method of claim 25, wherein the method is completed in about 1 hour to 24 hours.

34. The method of claim 31, wherein the sample contained less than 1,000 cfu of the pathogen to be detected.
